(19) United States
(12) Patent Application Publication
(10) Pub. No.: US 2009/0181344 A1
(43) Pub. Date: Jul. 16, 2009

(54) PHARMACEUTICAL COMPOUND TO PREVENT AND TREAT FOCAL TISSULAR LESIONS AND INFECTIONS, METHOD AND APPLICATORS

(76) Inventor: Ricardo Levisman, Buenos Aires (AR)

Correspondence Address: GREER, BURNS & CRAIN, 300 S WACKER DR, 25TH FLOOR, CHICAGO, IL, 60606, US

(21) Appl. No.: 12/360,606

(22) Filed: Jan. 27, 2009

Related U.S. Application Data
(63) Continuation-in-part of application No. 11/956,114, filed on Dec. 13, 2007.

(51) Int. Cl. A61C 3/00; A61C 5/00; A61K 33/30

(52) U.S. Cl. 433/164; 433/228.1; 424/643

(57) ABSTRACT

A therapeutic compound, methods and applicators to prevent and treat focal tissular lesions and infections in mammals is made by the mixture of an antiseptic powder and an antiseptic liquid, wherein the compound may be employed for an assisted integration of implants to bone, through the bone socket arrangement pre or post-implantation by medication in a patient, for preventing or curing by direct contact diseases within the mouth and also to treat skin inflammations, infections, aphthas, herpes simplex infections, ulcers, burns, and other externally illness, to promote healing and bone growth and to chemical debridement.

This application is a Continuation-In-Part of application Ser. No. 11/956,114, filed Dec. 13, 2007 and claims priority under 35 USC §120.

### BACKGROUND OF THE INVENTION

1. Field of the Invention

The present invention relates to a compound, methods and devices for use in the medical field to treat and prevent focal tissular lesions and infections in mammals, more particularly refers to a compound for preventing and treating focal tissular lesions and infections in the mouth and skin, and most particularly in dental implantology.

2. Description of the Prior Art

Thousands of patients are nowadays receiving oral and maxillofacial implants, and the number of beneficiaries is increasing more and more. This shows not only the degree of popularity, but also the technical advance that meets the demands of dentist surgeons, prosthodontists and patients.

Branemark and collaborators were the designers of the first modern submerged implant in line with their true theory about osseointegration. Such state is a balanced and harmonic coexistence between the bone and a rigid biocompatible element with direct and frictional-close contact for a long-term.

Osseointegration, although not a genuine solder, is clinically considered as a rigid fixation of the implant within the bone and under asymptomatic conditions.

The two-stage procedure, the removal of titanium native oxide, the meticulous sterilization and the four-month period waiting before loading the implant, were conditions included in the original protocol.

Later, the picture changed completely when it was demonstrated that the use of a submerged implant is not an imposition to achieve osseointegration. In addition there is a proof that a newly-inserted implant may be loaded without delay and that the placement of a non-submerged implant immediately after a tooth extraction is feasible and possible.

But an even bigger challenge is to successfully place an implant inside an alveolus that has been infected as a consequence of periapical and/or periodontal processes of a tooth to be removed.

However fallible, today either the one or the two-stage system is used indistinctly and both show that the issue is always related to avoiding the microorganisms action.

Implant immobility is a biologically imposed requirement in order to avoid its immediate failure. Primary stability is obtained by the highest possible adaptation between the implant and the bone. The compactness of the hard tissue around an implant inserted into an undersized bore by applying torque-force in apical direction guarantees its immobility, even in poor density bone.

With the pass of several hours, the bone flexibility facilitates the decrease of the pressure that the newly placed implant exerts against the socket wall and in consequence the ischemia as well.

The bone reacts to similar injuries in different ways, because it does not have a regular, uniform and stable structure. So, there exists the possibility that an implant with a very good initial stability does not later accomplish the integration with the bone. The early stability can be obtained even if bacteria infiltrate the socket. However, the bone bore must be aseptic to achieve the osseointegration.

Undoubtedly, at present there exist new requirements in implantology, for example it does not feature the same practical value the insertion of an implant using a basic technique some time after the extraction of a tooth that the replacement of same immediately after the removal with or without the application of grafting materials and besides with an instant loading.

Even though the percentage of failure has remarkably been reduced, it would be improper to assure the complete success up to now.

It is very important to employ an implant system which guarantees success at least in its initial stability, more engagement with soft and bone tissues, any assistance to better resist immediate loading and capacity to preserve a stable fixation even while using short-implants, imperfect bone quality and/or quantity.

There are many dental implant designs, most of them featuring specific morphological characteristics and special treatment of the outer surface to promote bone growth.

The expression “healing” may be best interpreted as the strict reparation of the injured soft and/or hard tissues, but the tissue grows as a process with visible increase of the mass. It is clear that the bone needs room to grow. The micro and macro gaps promote bone growth. The young bone advances within the implant pores, holes and grooves.

Bone healing comprises four steps as following: 1) clotting phase: the clot takes place on the wound in order to obtain hemostasis or stop blood loss. Platelets which integrate the clot by themselves sticks to form a mass that secretes inflammatory factors; 2) inflammatory phase: the intact older vessels of the wounded area increase its thickness to receive more leucocytes, limphocytes, fibroblasts and nutrients, and anastomosis of them with new vessels is produced, 3) secretory phase: fibroblasts rapidly secrete extracellular matrix of collagen type III, promote the development of substantially new capillaries and osteoblasts. The granulation tissue is composed by the preceding components plus immune cells. Fibroblasts activity demands oxygen, which is provided by haemoglobin; 4) maturation phase: fibroblasts changes the matrix of collagen type III into a stronger collagen type I and after that, osteoblasts complete the osteoid tissue formation. Further, osteoid need the same osteoblasts for its mineralization to become bone. Fibroblasts decrease in number when the matrix of collagen type I formation is done. The absence of haemoglobin due to the lack of a way to approach the oxygen to the damaged area may for example be replaced by a drug which cedes this element through a chemical reaction.

The osseointegration involves two different types of surfaces: the socket wall within the bone and the metallic implant; being the former organic and the latter inorganic. So far, most techniques have been developed to exclusively treat the implant outer surface before inserting the fixture and without considering the supply of the socket wall important as well. If the implant that has always been used by surgeons is a mechanized piece obtained from a titanium bar, with a prepared surface an uncontaminated neither by organic nor by inorganic matter, the failure of the bone and the implant integration obviously involves only the blood clot and the socket wall. Thus, after the above deduction, this inventor has developed what he calls: “assisted integration” of implants to bone, through the “bone socket arrangement”, pre or post-implantation, by medication as an independent and advanced procedure.

It is important to understand the reason to use this new step in all the cases, and if not, at least when an implant is inserted immediately after a tooth extraction and more particularly in the case of an implantation within the alveolus of a dental piece removed due to its contamination by polymicrobes.

The bacteria are able to invade the field where the clot will be formed, the blood that fills the bone socket, the clot directly or the three together. The analysis of the process evolution since the bacteria irruption into the clot is the following: bacteria attack the clot and are strongly entrapped and immobilized by the fibrils of the matrix. First, the anaerobic bacteria remain within the clot and the aerobic subsist on the clot surface. Both groups and others, live together in the same physical habitat called “community”. The survival of aerobic and anaerobic bacteria within the community is possible because the former consume the oxygen that is toxic to the second group. Immune response decreases if biofilm community matures.

If the best solution is always based on the prevention of any disease development, it is absolutely necessary to treat the drilled bore, because the gum and the bone are cut by a drill that exposed parts of a common wound on soft and hard tissues such as blood, minute vessels, fibroblasts, lacunas, cannaliculies, trabeculaes, osteocytes, osteoblasts, osteoclasts, etc. This is another reason why the bore wall needs some tool to be cauterized, disinfected and cleaned and also added nutrients in order to increase the cell size and its rapid division to repair damage.

After proving that the blood clot which fill the gaps between the socket wall and the implant may not always end up unharmed in its physiologic process of transformation into bone, this applicant discovered, through accurate proofs, that in order to level the certain results it is necessary to avoid the clot formation.

One conclusion assures that the bone and the gum effort should concentrate primarily on the healing process and not on controlling the bacteria action at the same time. If by themselves they could freely perform only the first function, the auxiliary support may be accomplished by synthetic drugs.

So, this “assisted-integration” of implants to bone through medication is a healing by second intention. In this case, the granulation tissue is generated directly by the outer layer of the periosteum and does not derivate from a clot as in the healing by first intention. Generally, during a conventional healing by second intention is used iodoform gauze or the known chirurgical cement to fill the socket. Both elements are useful, but not in contact with the surface of titanium implant.

The “assisted-integration” differs in part from a true healing by second intention, because it includes an uncontaminated implant inserted in the middle of the socket.

The inventor's attention was focused on, out of need, searching for the fit chemical formula so as to avoid the clot formation by occupying its place; destroy those bacteria that even when blocked, still infiltrate the wall of the bore during the implant insertion and/or the bacteria that infect the socket of a tooth with periapical and/or periodontal processes. Simultaneously, it must arrange the socket without attacking the normal cells, nor contaminating the implant surface, but with an additional skill in order to promote in due time by itself, the replacement of its mass by granulation tissue. The tact of avoiding completely the clotting phase and partially the inflammatory phase obligates to restore all the functions accomplished during the replaced phases by a special compound. Such compound must also fill the free cavity of a periapical and/or periodontal processes, where the infected tissue was previously removed by curettage immediately after the tooth extraction.

It is well known that during normal healing process, the human body gradually lyzes the clot in order to form granulation tissue. In the case of the inventive compound, the body, instead of lyzing the clot, actuates directly to phagocyte the medication to replace it by granulation tissue.

If a clot is infected by bacteria that assemble from their permanent living in the mouth, it is transmuted to pathological residues. A few days or weeks after contracting the infection, implant mobility appears in spite of its initial firmness; such disease is called “early periimplantitis”. The degree of implant micro-motion is directly proportional to the thickness of the pathological tissue grown around its surface.

The thickness of the tissue formed between the outer surface of a newly-placed implant and the inner wall of the alveolus, will be greater and in consequence also its mobility, because the implant diameter is generally smaller than the tooth socket. In this instance, the initial stability that an implant needs is obtained by a change in the alveolus direction, through a short new undersized bore drilled in the distal portion of the socket. So, the inevitable spaces between the implant and the bone wall will be filled by blood and then by the clot.

The implant rejection always comes from a local infection and occurs chiefly in the cases when an implant immediately replaces a dental root.

The above indicates that an implant, even with the best retention means on the outer surface, such as a porous surface, textured thread, macro-retentions, micro-retentions or hydroxyapatite coating, by themselves do not guarantee the achievement of the osseointegration.

No metal that produces toxic oxide on its outer surface can live with biological tissues. There are few biocompatible metals available, but titanium is preferably used to manufacture implants.

Titanium generates its own oxide layer from elements that yield oxygen, like water, salts, bone, etc. and such oxide does not per se has antibacterial properties.

If the implant oxidation is not a precondition to bone integration, this indicates an absence of implicit chemical association between the titanium oxide and the bone.

Periimplantitis involves five elements that may be contaminated by bacteria: implant, gum, bone, clot or pathological tissue. The symptoms and signs of early-periimplantitis are evidenced some time after implantation, because toxins secreted by bacteria next and around the implant need a short period of incubation before causing damage. When the perforation of the bone has been performed without taking into account the appropriate refrigeration, the resulting necrotic tissue is also rapidly invaded by bacteria.

To disclose the heterogeneous structure of the tissue formed from an infected clot, it was necessary to remove mobile implants to immediately collect by curettage fragments from the socket walls to carry out the histopathological and the bacteriological culture analysis. The result of these studies showed a large number of colonies of different species of bacteria dispersed along a fibrinocellular exudate.

Integrating the bacteria “community”, we found the anaerobic grampositive, which are the most harmful bacteria for the bone. Anaerobes are opportunistic pathogens and always associated to diseases with tissue exposure. They cause the formation of purulent abscess and can survive to air for long periods, but without growing.

The connective tissue of the periosteum outer layer produces a lot of undifferentiated cells which become osteoblasts if the growth factor secreted by fibroblasts activates the process. So, the fibroblasts and then the osteoblasts convert the clot into osteoid tissue, which is composed of a collagen matrix that must be mineralized by the same osteoblastst to become bone. There is strong evidence that bacteria can invade the osteoblast structure. If the cells, which phagocytes bacteria are not able to protect adequately the osteoblasts integrity, the process of bone formation remains unfinished. Anaerobes can persist inside the intracellular environment of osteoblasts and also survive within the dead cells keeping the capacity to attack other osteoblasts and increase the infection.

Autolysis is the dissolution of the necrotic tissue of a wound by the body own enzymes. Such physiological process is not as fast as the surgical debridement and neither active as the chemical debridement, which further can lyse the dead bacteria residues. The osteolysis mechanism is not well established yet.

Early-periimplantitis and alveolitis are similar disorders, but originated in different circumstances. Alveolitis is an acute infection of the alveolus, produced by the infiltration of bacteria inside the clot formed after tooth extraction, and its treatment may be carried out with the same curative product as the one used for periimplantitis.

The systematic “bone socket arrangement”, pre or post-implantation, by medication assures a stable alternative to avoid the early-periimplantitis and simultaneously to promote tissue healing and bone growth.

The undesirable consequence of using a treatment with limited action may be the establishment of a vicious circle, because bacteria colonies remaining from uncompleted previous decontamination can easily reinstall the disease. Bacteria increase their resistance from mutation changes. Moreover, biofilm which is a membrane of protein layer self-formed by the same bacteria, acts as an additional barrier for its own protection.

In contrast to gram-positive bacteria which possess only one membrane, gram-negative have two different and asymmetric membranes of lipids; the inner of phospholipids and the outer of lipopolysaccharides. The synthesis of the lipids occurs inside the inner membrane and they are transported to the outer membrane. The soluble protein secretion of gram-negative must go through both membranes. It is very difficult to penetrate such supplementary covers, even by local drugs.

The indiscriminate use of antibiotics to treat diseases, produce the gradual bacteria insensitivity against these drugs, and that demands each time the employ of higher doses to obtain the same results. The researchers are continuously developing more effective compounds to infiltrate new biofilms.

The prevention or fast treatment of mouth diseases is relevant because there is a potential risk of transmigration of the micro-organisms that might be dragged by the blood-stream, from activated septic focuses of the oral cavity to other different tissues.

In this regard, it is well known that phatogenic bacteria from bottom pockets have direct access to necrotic capillaries and, later, an easy passage to large vessels like arteries and veins. The new bacteria settlement, that results from own contagion is the cause of critical infections, such as endocarditis, cardiac valve septicity and on retropharingeal, endocraneal or osteoarticular tissues, etc.

Periimplantitis and mucositis pockets constitute chronic focuses of infection around dental implants. Typically, gingivitis as a reversible inflammation is manifested by spontaneous pain due to hyperemia, which facilitates the carrying of more cells in order to oppose resistance to germs activity.

Gingivitis, even without bone loss and pocket, still disseminates bacteria from the gingival sulcus. If the germs continue their degradation process on gingivitis, a physiological sulcus turns into a pathological pouch.

The progressive degradation of the osseointegration also occurs because certain fragility and vulnerability shows the field of union between the bone and the implant.

When a mobile implant is removed, it looks darker than the colour of pre-implantation, due to organic contamination. Such contamination contains living and dead bacteria, also residual secretion of their metabolism.

The inventor has carried out investigations to study the organic contamination on titanium and the composition of such contaminant products.

The tests began with several samples of titanium, previously decontaminated from organic and inorganic matter and introduced within a sterile culture medium of agar-agar with blood and other nutrients for some time; when they were examined they showed absence of contaminants. Then, equal samples, prepared in the same form, were submerged into a culture medium, but this time contaminated with living mouth flora and maintained in a stove at a continuous temperature of 37° C. (98.60° F.) for a month.

The results showed clearly that bacteria produce contamination on the aseptic titanium. This was confirmed with an infrared spectrometer machine. The spectroscopy on titanium samples, which were introduced within a culture medium contaminated only with anaerobic gram-positive bacteria, showed a layer of lipids.

If the dynamic and uncontrollable process of contamination continues from the proximal portion of the implant to the distal end, it puts the osseointegration on stake. Each new impure film produced by bacteria is added to the previous one and the amount separates the metal more and more from the bone; the result is bone resorption.

The osseointegration takes place directly between the bone and the titanium or between the bone and any permanent and bioactive coating layer which may surrounds the implant.

Synthetic hydroxyapatite is a biocompatible mixture of salts, which is used by surgeons to fill defects in bone, because, without changes, it can replace the HA that integrates biologically the bone structure. Because it has affinity with living hard tissue, it is also used to cover, with a thin layer, the outer surface of a titanium implant so as to induce cohesion with the bone. This interposed element avoids the direct contact between the metal and the bone, but often the apparent initial benefit contrasts with the final result, when the implant fails due to the detachment of the HA from the titanium.

At present, some implant factories are still looking for a way to adhere HA to titanium, which so far has not been well solved. Definitely, such material is not essential to achieve osseointegration.

Many years after implantation, an osseointegrated implant with or without loading can also acquire “late-periimplantitis”, principally caused by bacteria invasion.

Implant over-work may also generate indirectly a disorder, though in fewer circumstances. The consequence of an excessive loading is reflected on the decrease of bone density around the implant. So, poor density bone around an implant entices bacteria to infiltrate the area.

The sequence of a late-periimplantitis is as follows: the cervical portion of a crown retained over the implant or the collar of the same implant head is first contaminated by bacteria plaque, immediately the gum suffers inflammation and changes its coloration and morphology, and may be accompanied or not with blood loss. If the edema is not reverted by local treatment and bacteria maintains the disturbing action, the process turns into mucositis, which is restricted to gingivitis associated with exudate. At the same time, the bone begins to receive the toxin effects and starts its own retraction.

Biologically, a tooth is attached to bone through a thin layer of fibers, which may be invaded by bacteria from the gingival sulcus. Hence, that destructive disorder called periodontitis is originated in a simple gingivitis. The progressive and whole evolution of periodontitis is the following: gingivitis, gingival sulcus defacement, formation of gingival pocket, progressive replacement of the periodontal ligament by pathological tissue, bone atrophy, osteomyelitis, suppuration, high tooth mobility, eventually an abscess and finally tooth loss.

Periimplantitis and periodontitis diseases feature similar characteristics but in a different context, but the bone in both cases is initially reabsorbed as a funnel and then horizontally. When periodontitis is not excessively advanced, it leads to use the same prescription as the one used for periimplantitis treatment. It is well known, that the incidence of periimplantitis and mucositis is higher in patients where implants replace teeth loss because of chronic periodontitis.

New alternative techniques to preserve gum and bone integrity are tested all the time, because the mouth is an open cavity, prone to develop bacteria colonies within teeth cavities, gingival sulcus, between teeth and on the tongue.

Different factors are added to conventional etiology, such as the patient hereditary predisposition, smoking and a lower immune protection.

It is possible to see for many years a stable implant fixed within the bone and only supported from the distal portion of the piece, with most of its body exposed in the mouth. Such illness, undesirably frequent, is called “inactive periimplantitis”, often without a prescribed treatment.

Fundamentally, the prevention of the harmful behaviour of bacteria consists in a meticulous cleaning of gum, teeth and tongue. The toothpaste is capable to assist in dental and gum hygiene, deodorizing, desensitizing, whitening, polishing with abrasives that are always included in its composition, but it is not qualified by itself to remedy the alveolitis, periimplantitis, mucositis or periodontitis.

Gingivitis, if in a lesser degree, requires rigorous oral hygiene as first help, plus a program to regularly apply therapeutics pomade locally.

If inflammation and/or infection around the implant also involve the tissues of adjacent teeth, the same treatment as the one to cure periimplantitis is applied.

It is also possible to choose one of the non-specific methods to treat periimplantitis, such as the abrasion of the implant surface with carbon particles, citric acid solution, antibiotics and laser surgery, etc.

### SUMMARY

It is therefore an object of the present invention to provide a compound exhibiting a broad cicatrizing action, ample antibacterial, antifungus and antiviral spectrum to prevent and treat focal tissue lesions and infections in mammals, the compound also providing the ability to fast chemical debridement of necrotic tissues and dead bacteria residues, ductility to fill cavities already free of any infected tissue, properties for an “assisted integration” of implants to bone, through the “bone socket arrangement”, pre or post implantation, and means to promote tissue healing, wherein the compound comprises:

a) an antiseptic liquid comprising, per each 100 millilitres thereof:

acetic acid, aqueous solution 6% v/v, in a range between 2 and 3 millilitres;

derivative of sulphonic acid, 100% v/v, in a range between 0.50 and 1.50 millilitres;

sodium phosphate tribasic, 100% p/p, in a range between 2 and 3 grams, and

distilled water, s.q. 100 millilitres, and

b) an antiseptic powder comprising, per each 100 grams thereof:

zinc oxide, 100% p/p, in a range between 15 and 25 grams;

alginic acid, 100% p/p, in a range between 10 and 20 grams;

carboximethyl-cellulose, 100% p/p, in a range between 10 and 20 grams;

gum arabic, 100% p/p, in a range between 15 and 25 grams;

magnesium oxide, 100% p/p, in a range between 5 and 15 grams; and

calcium citrate, 100% p/p, in a range between 15 and 25 grams,

It is another object of the present invention to provide an antiseptic liquid comprising per 100 millilitres thereof:

acetic acid, aqueous solution 6% v/v, 2.50 millilitres;

derivative of sulphonic acid, 100% v/v, 1 millilitre;

sodium phosphate tribasic, 100 p/p, 2.50 grams; and

distilled water, s.q. 100 millilitres.

It is still another object of the present invention to provide an antiseptic powder comprising, per 100 grams thereof:

zinc oxide, 100 p/p, 20 grams;

alginic acid, 100% p/p, 15 grams;

carboximethyl-cellulose, 100 p/p, 15 grams;

gum arabic, 100 p/p, 20 grams;

magnesium oxide, p/p, 10 grams; and

calcium citrate, 100% p/p, 20 grams.

It is still another object of the present invention to provide a therapeutic compound to kill bacteria anaerobic and aerobic, gram-positive and gram-negative.

It is still another object of the present invention to provide a method for obtaining a therapeutic paste, wherein the method comprises the step of mixing the antiseptic liquid and the antiseptic powder with a sterile spatula over a sterile glass slab.

It is still another object of the present invention to provide a therapeutic compound in the form of pomade form.

It is another object of the present invention to replace a clot by the compound.

It is another object of the present invention to provide a compound with ductility to fill cavities of periapical and/or periodontal processes, during the implantation procedure. Such cavities already free of any infected tissue, which must be removed by curettage immediately after the tooth extraction.

It is another object of the present invention to provide a resorbable paste.

It is another object of the present invention to provide a biocompatible paste.

It is another object of the invention to provide a paste capable of fast chemical debridement and also of lyses of dead bacteria residues.

It is still another object of the present invention to provide devices or applicators to carry the therapeutic compound.

It is a further object of the present invention to provide a method for obtaining an antiseptic liquid comprising, per each 100 millilitres thereof:

acetic acid, aqueous solution 6% v/v, in a range between 2 and 3 millilitres;

derivative of sulphonic acid, 100% v/v, in a range between 0.50 and 1.50 millilitres;

sodium phosphate tribasic, 100% p/p, in a range between 2 and 3 grams; and

distilled water, s.q. 100 millilitres, the method comprising the steps of:

a) measuring by means of pipettes the volume of the acetic acid, the derivative of sulfonic acid and the sterile distilled water;

b) adding the acetic acid, the derivative of sulphonic acid to the water,

c) shaking the components of steps b);

d) weighing with a scale the sodium phosphate tribasic;

e) adding the sodium phosphate tribasic to the solution, and

f) shaking the components of steps b) and e).

It is a further object of the present invention to provide a method to prepare an antiseptic powder comprising, per each 100 grams thereof:

zinc oxide, 100% p/p, in a range between 15 and 25 grams;

alginic acid, 100% p/p, in a range between 10 and 20 grams;

carboximethyl-cellulose, 100% p/p, in a range between 10 and 20 grams;

gum arabic, 100% p/p, in a range between 15 and 25 grams;

magnesium oxide, 100% p/p, in a range between 5 and 15 grams;

calcium citrate, 100% p/p, in a range between 15 and 25 grams;

the method comprising the steps of:

a) weighing with scale the zinc oxide, the alginic acid, the carboximethyl-cellulose, the gum arabic, the magnesium oxide and the calcium citrate; and

b) shaking the components of step a) in a shaker until obtaining homogeneity.

It is a further object of the present invention to provide a method for obtaining a compound exhibiting a broad cicatrizing, ample antibacterial, antifungus and antiviral spectrum to prevent and treat focal tissue lesions and infections in mammals, also capable of fast chemical debridement of necrotic tissues and dead bacteria residues, ductility to fill cavities already free of any infected tissue, properties for an “assisted integration” of implants to bone, through the bone socket arrangement, pre or post-implantation by medication, and means to promote tissue healing, wherein the compound comprises:

a) an antiseptic liquid comprising, per each 100 millilitres thereof:

acetic acid, aqueous solution 6% v/v, in a range between 2 and 3 millilitres;

derivative of sulphonic acid, 100% v/v, in a range between 0.50 and 1.50 millilitres;

sodium phosphate tribasic, 100% p/p, in a range between 2 and 3 grams; and

sterile distilled water, s.q. 100 millilitres, and

b) an antiseptic powder comprising, per each 100 grams thereof:

zinc oxide, 100% p/p, in a range between 15 and 25 grams;

alginic acid, 100% p/p, in a range between 10 and 20 grams;

carboximethyl-cellulose, 100% p/p, in a range between 10 and 20 grams;

gum arabic, 100% p/p, in a range between 15 and 25 grams;

magnesium oxide, 100% p/p, in a range between 5 and 15 grams;

calcium citrate, 100% p/p, in a range between 15 and 25 grams.

and wherein the method comprises the step of:

mixing the antiseptic liquid with the antiseptic powder with a sterile spatula over a sterile glass slab to obtain a paste.

It is another object of the present invention to provide a compound applicator to administer the therapeutic compound onto anaphtha.

It is a further object of the present invention to provide a compound applicator for applying the therapeutic compound onto a wound, wherein it is in a form of a gauze or and adhesive bandage.

It is still another object of the present invention to provide a compound applicator, wherein it is in a stem form.

It is still another object of the present invention to provide a compound applicator, wherein it is a stem with different geometric shapes, such as cylindrical, conical, polygonal, etc.

It is still another object of the present invention to provide a compound applicator, wherein it is a smooth stem, with retention means or both together.

It is still another object of the present invention to provide a compound applicator made of plastic, metal or wood.

It is still another object of the present invention to provide a method to arrange the bone socket immediately after the extraction of a dental piece; wherein the method comprises the steps of:

a) providing the removable compound applicator in stem form;

b) taking the compound in a soft-paste form;

c) coat-by-smearing the paste in a layer over the applicator;

d) inserting the applicator with the compound into the bone socket;

e) waiting for a period of time; and

f) removing only the compound applicator.

It is still another object of the present invention to provide a method for bone socket arrangement immediately after a dental piece has been removed, the method comprising the steps of:

a) providing a removable compound applicator in plug form;

b) mixing the antiseptic liquid and the antiseptic powder of the invention with a sterile spatula over a sterile glass slab;

c) coat-by-smearing the soft-paste in a layer over the applicator;

d) inserting the applicator with the compound into the socket;

e) waiting for a period of time, preferably between about 9 minutes and about 30 minutes, most preferably 15 minutes; and

f) removing the compound applicator.

It is even another object of the present invention to provide a method for applying the inventive compound onto a wound, wherein the method comprises the steps of:

a) providing an applicator taking the form of a gauze or an adhesive bandage;

b) placing onto a wound the applicator with the compound,

c) waiting a period of time, and

d) removing the applicator.

It is still another object of the present invention to provide a method for coat-by-smearing a layer of soft-paste around the outer surface of a dental implant immediately before the implantation.

It is another object of the present invention to provide a layer of therapeutic compound around the outer surface of a dental implant, coated a period of time before its use, wherein the hardened paste needs to become active by means of its moistening only with the liquid of the compound immediately before the implantation.

It is still another object of the present invention to provide a method to apply the compound onto a wound, wherein the method comprising the steps of:

a) providing the compound applicator in the form of one of a gauze or adhesive bandage;

b) taking the compound in soft-paste form;

c) coat-by-smearing the soft-paste in a layer over the applicator;

d) placing the applicator onto the wound for a period of time, and

e) removing only the applicator.

It is a further object of the present invention to provide a compound applicator to apply the therapeutic compound of the invention onto anaphtha, wherein such applicator comprises a stem.

It is yet another object of the present invention to provide a compound applicator for use with the inventive compound and methods, wherein the applicator may be polygonal, conical, made of wood, metal or plastic, with retention means, smooth, rigid, flexible, in the form of a non-removable plug or removable plug.

It is a further object of the present invention to provide a method for bone socket arrangement, post-implantation, in a patient, by using the compound of the invention, wherein the method comprises the steps of:

a) providing a dental implant, decontaminated from organic and inorganic matter;

b) mixing the antiseptic liquid and the antiseptic powder with a sterile spatula over a sterile glass slab to obtain a soft-paste;

c) coat-by-smearing the soft-paste over the implant, and

c) inserting the dental implant with the compound into the socket in a permanent form.

It is yet another object of the present invention to provide a method for an “assisted integration” of implants to bone, through the “bone socket arrangement”, pre-implantation, in a patient, by using the compound of the invention, the method comprising the steps of:

a) providing a removable compound applicator in the form of a plug;

b) mixing the antiseptic liquid and the antiseptic powder with a sterile spatula over a sterile glass slab to obtain a soft-paste;

c) coat-by-smearing the soft-paste over the applicator;

d) inserting the applicator with the compound into the bone socket;

e) waiting for a period of time; and

f) removing the applicator from the bone socket.

It is yet another object of the present invention to provide an applicator carrying the compound oft the invention, in a form of hardened paste that is moistened with the liquid of the inventive compound immediately before the application of the compound in a patient.

It is still another object of the present invention to provide a therapeutic compound with a biological PH range.

It is still another object of the present invention to provide a therapeutic compound, wherein it may be stored as a kit, composed by an antiseptic liquid and an antiseptic powder, each one contained in its package.

It is still another object of the present invention to provide a therapeutic means, wherein it may be stored independently as an antiseptic liquid or as an antiseptic powder.

It is still another object of the present invention to provide a therapeutic compound, wherein it is in paste form, either in soft-paste or hard-paste.

It is still another object of the present invention to provide a therapeutic paste, wherein it must be soft to feature effectiveness.

It is still another object of the present invention to provide a removable conical stem applicator to be stored with a layer of therapeutic compound coated a period of time before its use, wherein the hardened paste needs to become soft by means of its moistening only with the liquid of the same compound immediately before the application.

It is still another object of the present invention to provide a new compound that constitutes another alternative in the medical field to treat and prevent focal tissular lesions and infections, wherein the prescription for such compound given by medical doctors, dermatologists, stomatologists and surgeons does not have specific restrictions, because its ingredients are all biocompatibles and the concentration used in each one of them has a nontoxic range. The referred compound was developed to act on wounds, aphthas, ulcers, and also to play an important role in the “assisted-integration” of implants.

### BRIEF DESCRIPTION OF THE DRAWINGS

In order to more fully understand the drawings referred to in detailed description of the present invention, a brief description of each drawing is presented, in which:

FIG. 1 is a cross section view of an applicator with a layer of the therapeutic compound extended along a conical stem, which is provided with a convex and a concave retention means according to a preferred embodiment of the present invention, and

FIG. 2 is a cross section view of an applicator with the compound in a hardened paste form on the top of the cylindrical stem, which is provided with a concave retention means according to another preferred embodiment of the present invention.

### DESCRIPTION OF THE PREFERRED EMBODIMENTS

The invention relates to a therapeutic compound exhibiting a broad cicatrizing, ample and residual antibacterial, antifungus and antiviral spectrum to prevent and treat focal tissular lesions and infections in mammals, also capable of fast chemical debridement of necrotic tissues and dead bacteria residues, ductility to fill cavities already free of any infected tissue, properties for an “assisted integration” of implants to bone, through the “bone socket arrangement” pre or post-implantation by medication and means to promote tissue healing.

Such biocompatible and resorbable compound must be a soft-paste to coat-by-smearing a layer over the outer surface of an implant before its insertion or over a compound applicator. The compound is used to avoid clot formation and in this way early-periimplantitis, protect the tissue healing, to increase initial stability of an implant and to benefit its immediate loading. The new step, called by the applicant: “assisted integration” of implants to bone, through the “bone socket arrangement”, pre or post-implantation by a compound that accomplish with the requirements that the bone socket demands as to be cauterized, disinfected, cleaned and provided with nutrients to encourage cell division.

The compound must fill cavities of periapical and/or periodontal processes during the implantation procedure. Such cavities already free of any infected tissue, which must be removed by curettage, immediately after the tooth extraction.

There are two forms of implementing the “bone socket arrangement” method: a) pre-implantation and b) post-implantation. The benefit of using one or the other procedure depends on a series of circumstances.

This pharmaceutical product does not contain antibiotics. The reason was founded on the fast detriment of its efficiency when it is used together with acids and due to the gradual bacteria insensitivity against antibiotics as to demand a higher dose of them to obtain the same results.

The therapeutic compound of the present invention comprises a mixture of an antiseptic liquid and an antiseptic powder.

Each 100 ml of the antiseptic liquid comprises:

acetic acid (aqueous solution 6% v/v) 2.50 millilitres;

derivative of sulfonic acid (100 v/v) 1 millilitre;

sodium phosphate tribasic (100 p/p) 2.50 gram;

and distilled water, s.q. 100 millilitres;

Each 100 grams of the antiseptic powder comprises:

zinc oxide (100% p/p) 20 grams;

alginic acid (100% p/p) 15 grams;

carboximethyl-cellulose (100 p/p) 15 grams;

gum arabic (100% p/p) 20 grams;

magnesium oxide (100% p/p) 10 grams;

calcium citrate, (100% p/p) 20 grams.

The present invention also provides a method to prepare both, the antiseptic liquid and the antiseptic powder. The antiseptic liquid method comprises the steps of:

a) measuring by means of pipettes the volume of the acetic acid, the derivative of sulfonic acid and the sterile distilled water;

b) adding the acetic acid and the derivative of sulfonic acid to the sterile distilled water;

c) shaking the liquids of step b);

d) weighing with scale the sodium phosphate tribasic;

e) adding the sodium phosphate tribasic to the liquid of step b); and

f) shaking the components of steps b) and e);

Both, the selected group of reagents and the buffers, must be dissolved and dispersed into sterile distilled water, which was chosen as solvent.

The method for obtaining the antiseptic powder comprises the steps of:

a) weighing with scale the zinc oxide, the alginic acid, the carboximethyl-cellulose, the gum arabic, the magnesium oxide and the calcium citrate; and

b) shaking the above components in a shaker until obtaining homogeneity.

The therapeutic compound of this invention is preferably prepared by the following method:

mixing 5 drops of the antiseptic liquid with 0.25 gram of the antiseptic powder with a spatula over a glass slab.

The biological PH value of this therapeutic compound is critical, because if it is too acid or alkaline it may injure tissues. The range of such PH may be adjusted in a physiological PH value to avoid damage on teeth, bone, mucous, gum, skin and tongue.

The therapeutic compound comprises some buffer agents to suit and maintain the desired PH factor.

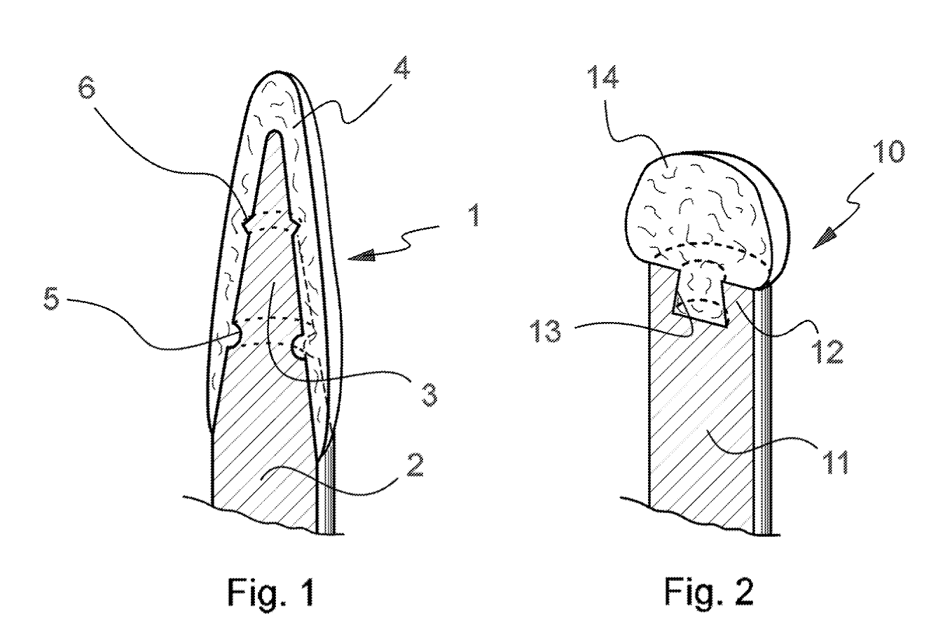

According to the embodiment of FIG. 1, the removable applicator is preferably indicated for applying the therapeutic compound within a bone socket and is illustrated with general reference as number 1. The applicator comprises a stem 2, made of any suitable material as plastic, metal or wood and has an upper end 3; the paste 4 is extended and adhered along the stem 2 at the end 3 inclusive. End 3 is preferably provided with retention means such as concave means, like a groove 5, or convex means like ribs 6, etc.

According to another embodiment of the invention, FIG. 2 shows an applicator illustrated by general reference 10, wherein applicator 10 comprises a body 11, also made of any suitable material like stem 1, and having an upper end 12 provided with retention means, preferably concave retention means such as a cavity 13. The inventive compound in the form of a paste 14 is fixed to end 12 through cavity 13. This embodiment was preferably designed to apply the inventive compound over wounds, aphtha, etc.

The present therapeutic compound has different but complementary ingredients. For a better understanding the individual function and the valuable properties of each one is hereafter given:

1) the zinc as a biological component of mammals body, is an essential element needed to support the body immune system, and also for the growth and mineralization of the body tissue. Zinc oxide was included in the compound for its natural conditions and to give volume to the powder;

2) the alginic acid, which has a very low PH is a hydrophilic colloidal polysaccharide and viscous gum, that is extracted from marine brown algae; it is used as a gelling agent, stabilizer, hemostatic, thickness and due to the condition to quickly absorb liquids is useful as slimming additive in dehydrated products;

3) the carboximethyl-cellulose is a soluble polymer, which through its hygroscopic property can absorb many liquids and slowly dispense them; also, it is useful as agglutinant and to confer adhesiveness to the paste;

4) the gum arabic was selected to increase the thin adhesiveness and to improve its disinflammatory property;

5) the magnesium oxide is useful as high alkaline PH corrector;

6) the calcium citrate is the calcium salt of the citric acid. It is used as a food additive, preservative and water softener, because the citrate ions can chelate with metal ions. Citrates are excellent buffers.

The liquid is a stable aqueous antiseptic solution and its composition is:

1) sterile distilled water as solvent;

2) acetic acid;

3) derivative of sulfonic acid; and

4) sodium phosphate tribasic.

The sodium phosphate tribasic is a powder ionic salt, highly water soluble and with an alkaline PH; dissolved in the liquid acts as buffering agent. In order to avoid the irritating and burning effects on the tissues, all the active drugs are also diluted in sterile distilled water. Sulpha drugs are antibacterial agents derived from some sulfonic acid. The cleaning function of the paste is attributed to sulfonic acid derivatives. Also its sulfonate salts are important as detergents, surfactants and to lower the interfacial tension between liquids.

After mixing the liquid and the powder, the components of the compound have pharmacological activity as zinc salts. All the acids reacts with the zinc oxide to forms salts of different solubility. According to the velocity of individual action or also of synthesis, the order of reaction is the following:

a) immediate action: alkylsulfonate of zinc;

b) quick action: zinc acetate;

c) intermediate action: zinc phosphate; and

d) slow action: zinc citrate chelate and remains zinc oxide. The remainder zinc oxide has an antibacterial and antifungus action by contact. Also, the calcium of the citrate is displaced by the zinc in benefit of the saliva PH and then slowly forms new portions of zinc citrate and zinc citrate chelate from the remainder zinc oxide.

Finally, the calcium and the phosphate are incorporated to bone mass.

This demonstrates the chemical addiction between the liquid and the powder of the compound. The compound effectiveness is given by the consistent drug-drug interaction.

The antimicrobial action of the compound was tested by means of a culture medium of agar-agar with nutrients and contaminated by all kinds of bacteria living in the mouth; so, the therapeutic paste in sphere forms of soft-paste freshly prepared was placed over the culture for a period of time. After that, a halo of inhibition of bacteria growth below the circle of the spheres was observed.

The result was unexpected when the therapeutic compound in sphere forms but, on this occasion, with the paste hardened days before its introduction into the infected culture medium for the same period of time, showed a small antimicrobial activity below the spheres. Nor was the paste effective when hardened spheres were softened with distilled water before the evaluation.

However, the answer was greater to the first experience when the spheres of hard-paste were softened by means of their moistening only with the liquid of the compound immediately before the in vitro proof.

The same medication shows excellent results when it is used to cure anaphtha or ulcer on the mucous membrane, the gum, the tongue and other diseases on the skin. The inventor has designed for each use an original element to sustain, carry and maintain the paste on the painful spot.

Definitely, the present therapeutic compound has a strong antibacterial action, also the ability to induce the coagulation of minute blood vessels and the chemical debridement of necrotic tissues conjunctly with the lyses of dead bacteria residues.

None of these ingredients stains, so the product does not colour adjacent natural teeth by contact.

The paste to treat skin injure is available: a) over an adhesive bandage and/or b) over gauze or similar.

The humidity of the mouth should lessen at least for fifteen minutes.

The body gradually excretes the residues of the resorbable paste without damaging organs.

While preferred embodiments of the present invention have been illustrated and described, it will be obvious to those skilled in the art that various changes and modifications may be made therein without departing from the scope of the invention as defined in the appended claims.

I claim:

1. A compound exhibiting a broad cicatrizing action, ample antibacterial, antifungus and antiviral spectrum to prevent and treat focal tissue lesions and infections in mammals, the compound also providing the ability to fast chemical debridement of necrotic tissues and dead bacteria residues, ductility to fill cavities already free of any infected tissue, properties for an “assisted integration” of implants to bone, through the bone “socket arrangement pre or post implantation”, and means to promote tissue healing, wherein the compound comprises:
a) an antiseptic liquid comprising, per each 100 millilitres thereof:
acetic acid, aqueous solution 6% v/v, in a range between 2 and 3 millilitres;
derivative of sulphonic acid, 100% v/v, in a range between 0.50 and 1.50 millilitres;
sodium phosphate trihasic, 100% p/p, in a range between 2 and 3 grams; and
distilled water, s.q. 100 millilitres, and
b) an antiseptic powder comprising, per each 100 grams thereof:
zinc oxide, 100% p/p, in a range between 15 and 25 grams;
alginic acid, 100% p/p, in a range between 10 and 20 grams;
carboximethyl-cellulose, 100% p/p, in a range between 10 and 20 grams;
gum arabic, 100% p/p, in a range between 15 and 25 grams;
magnesium oxide, 100% p/p, in a range between 5 and 15 grams;
calcium citrate, 100% p/p, in a range between 15 and 25 grams.

2. The compound of claim 1, wherein the antiseptic liquid comprises, per 100 millilitres thereof:
acetic acid, aqueous solution 6% v/v, 2.50 millilitres;
derivative of sulphonic acid, 100% v/v, 1 millilitre;
sodium phosphate tribasic, 100 p/p, 2.50 gram; and
distilled water, s.q. 100 millilitres.

3. The compound of claim 1, wherein the antiseptic powder comprises, per 100 grams thereof:
zinc oxide, 100 p/p, 20 grams;
alginic acid, 100% p/p, 15 grams;
carboximethyl-cellulose, 100 p/p, 15 grams;
gum arabic, 100 p/p, 20 grams;
magnesium oxide, 100% p/p, 10 grams; and
calcium citrate, 100% p/p, 20 grams.

4. The compound of claim 1, wherein it is in a soft-paste form prepared immediately before the use thereof by mixing the antiseptic liquid and the antiseptic powder with a spatula over a glass slab.

5. The compound of claim 1, wherein it is in a hard-paste form carried on an active portion of an applicator.

6. The compound of claim 5, wherein the hard-paste is softened immediately before the use by moistening it with the antiseptic liquid.

7. A method for obtaining a compound exhibiting a broad cicatrizing, ample antibacterial, antifungus and antiviral spectrum to prevent and treat focal tissue lesions and infections in mammals, also capable of fast chemical debridement of necrotic tissues and dead bacteria residues, ductility to fill cavity already free of any infected tissue, properties for an “assisted integration” of implants to bone, through the “bone socket arrangement”, pre or post-implantation, and means to promote tissue healing, wherein the compound comprises:
a) an antiseptic liquid comprising, per each 100 millilitres thereof:
acetic acid, aqueous solution 6% v/v, in a range between 2 and 3 millilitres;
derivative of sulphonic acid, 100% v/v, in a range between 0.50 and 1.50 millilitres;
sodium phosphate tribasic, 100% p/p, in a range between 2 and 3 grams; and
sterile distilled water, s.q, 100 millilitres, and
b) an antiseptic powder comprising, per each 100 grams thereof:
zinc oxide, 100% p/p, in a range between 15 and 25 grams;
alginic acid, 100% p/p, in a range between 10 and 20 grams;
carboximethyl-cellulose, 100% p/p, in a range between 10 and 20 grams;
gum arabic, 100% p/p, in a range between 15 and 25 grams;
magnesium oxide, 100% p/p, in a range between and 15 grams; and
calcium citrate, 100% p/p, in a range between 15 and 25 grams;
and wherein the method comprises the step of:
mixing the antiseptic liquid with the antiseptic powder with a sterile spatula over a sterile glass slab to obtain a paste.

8. A compound applicator for inserting the therapeutic compound of claim 1 into a bone socket, wherein such compound applicator is a removable plug.

9. The compound applicator of claim 8, wherein it is an applicator made of a material selected from the group consisting of wood, metal and plastic.

10. A method for “bone socket arrangement” immediately after a dental piece has been removed, the method comprising the steps of:
a) providing a compound applicator;
b) mixing the antiseptic liquid of claim 2 and an antiseptic powder comprising, per 100 grams thereof:
zinc oxide, 100 p/p, 20 grams;
alginic acid, 100% p/p, 15 grams;
carboximethyl-cellulose, 100 p/p, 15 grams;
gum arabic, 100 p/p, 20 grams;
magnesium oxide, 100% p/p, 10 grams; and
calcium citrate, 100% p/p, 20 grams, with a sterile spatula over a sterile glass slab;
c) coat-by-smearing the soft-paste in a layer over the applicator;
d) inserting the applicator with the compound into the socket;
e) waiting for a period of time; and
f) removing the compound applicator.

11. The method of claim 10, wherein the waiting period of time is between about 9 minutes and about 30 minutes.

12. A compound applicator to administer the compound of claim 1 onto a wound, wherein such compound applicator is a gauze.

13. A compound applicator to insert the therapeutic compound of claim 1 onto a wound, wherein such compound applicator is an adhesive bandage.

14. A method for applying the compound of claim 1 onto a wound, wherein the method comprises the steps of:
a) providing an applicator taking the form of one of a gauze or an adhesive bandage;
b) placing onto a wound the applicator with the compound of claim 1,
c) waiting a period of time, and
d) removing the applicator.

15. A compound applicator to apply the therapeutic compound of claim 1 onto anaphtha, wherein such applicator comprises a stem.

16. The compound applicator of claim 15, wherein it is a stem made of a material selected from the group consisting of wood, metal and plastic.

17. The compound applicator of claim 15, further comprising retention means.

18. A compound applicator for inserting the therapeutic compound of claim 1 into a bone socket, wherein such applicator is a non-removable plug.

19. A method for administering the compound of claim 1 to a patient, wherein the compound is in a soft-paste form and it is coat-by-smearing method over the applicator selected from one of a removable and non-removable plug.

20. A method for “bone socket arrangement”, post-implantation of implants, in a patient, by using the compound of claim 1, the method comprising the steps of:
a) providing a dental implant, decontaminated from organic and inorganic matter;
b) mixing the antiseptic liquid and the antiseptic powder of claim 1 with a sterile spatula over a sterile glass slab to obtain a soft-paste;
c) coat-by-smearing the soft-paste over the implant, and
d) inserting the dental implant with the compound into the bone socket in a permanent form.

21. A method for “bone socket arrangement”, pre-implantation of implants, in a patient, by using the compound of claim 1, the method comprising the steps of:
a) providing a compound applicator;
b) mixing the antiseptic liquid and the antiseptic powder of claim 1 with a sterile spatula over a sterile glass slab to obtain a soft-paste;
c) coat-by-smearing the soft-paste over the applicator;
d) inserting the applicator with the compound into the bone socket;
e) waiting for a period of time; and
f) removing only the applicator from the bone socket.

22. An applicator carrying the compound of claim 1 in a form of hardened paste that is moistened with an antiseptic liquid comprising, per 100 millilitres thereof:
acetic acid, aqueous solution 6% v/v, 2.50 millilitres;
derivative of sulphonic acid, 100, v/v, 1 millilitre;
sodium phosphate tribasic, 100 p/p, 2.50 gram; and
distilled water, s.q. 100 millilitres immediately before the application of the compound in a patient.

23. The compound of claim 1, wherein it is in a pomade form.

24. The compound of claim 1, wherein said compound is resorbable.
